(19) United States
(12) Patent Application Publication
(10) Pub. No.: US 2005/0106149 A1
(43) Pub. Date: May 19, 2005

(54) Antibodies abolish prion propagation and remote clearance of infectivity

(76) Inventors: Stanley B. Prusiner, San Francisco, CA (US); David Peretz, San Francisco, CA (US); R. Anthony Williamson, San Diego, CA (US); Dennis R. Burton, La Jolla, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/475,558

(22) PCT Filed: Apr. 25, 2002
(86) PCT No.: PCT/US02/13346

Related U.S. Application Data
(60) Provisional application No. 60/287,971, filed on May 1, 2001.

(51) Int. Cl. A61K 39/395

(52) U.S. Cl. 424/146.1

(57) ABSTRACT

Methods are disclosed whereby formulations of molecules are administered rendering cells resistant to infection with infectious proteins such as prions. The formulation preferably comprises a plurality of Fab fragments which (1) recognize and selectively bind to a range of epitopes on the protein of interest, (e.g. epitopes on PrP^{C}) and (2) bind to epitopes which interrupt the chain of events resulting in a change of the protein's conformation to an infectious disease conformation of the protein. The molecules and formulation are also useful in clearing infectious proteins from cells.

### STATEMENT REGARDING FEDERALLY SPONSORED RESEARCH

This invention was made with government support under grant nos. NS14069 and AG02132 awarded by the National Institutes of Health. The government may have certain rights in this invention.

### FIELD OF THE INVENTION

The present invention relates generally to therapeutics and methods of treatment and more particularly to therapeutics and methods of treating or preventing infections with infectious proteins such as prions.

### BACKGROUND OF THE INVENTION

In the favored model of prion replication, direct interaction between a pathogenic prion protein (PrP^{Sc}) template and an endogenous cellular PrP (PrP^{C}) protein is proposed to drive the formation of nascent infectious prions. (Telling, G. C. et al. Evidence for the conformation of the pathologic isoform of the prion protein enciohering and propagating prion diversity. Science 274, 2079-2082 (1996); Prusiner, S. B. Prions. Proc. Natl Acad. Sci USA 95, 13363-13383 (1998).) Reagents specifically binding either PrP conformer have the potential to interrupt prion production by inhibiting this interaction.

Concern over the continuing emergence of variant Creutzfeldt-Jacob disease, which is closely associated with exposure to bovine spongiform encephalopathy prions, has escalated the need for effective prion therapeutics. (Brown, P., Will, R. G., Bradley, R., Asher, D. M., and Detwiler, L. Bovine spongiform encephalopathy and variant Creutzfeldt-Jakob disease: Background, evolution, and current concerns. Emerging Infectious Diseases 7, 6-16 (2001); Bruce, M. E. et al. Transmissions to mice indicate that ‘new variant’ CJD is caused by the BSE agent. Nature 389, 498-501 (1997)) Prion propagation is thought to proceed via a mechanism involving specific interaction between PrP^{C} and PrP^{Sc}, possibly in the presence of an additional cellular factor. (Prusiner, S. B. Prions. Proc. Natl Acad. Sci USA 95, 13363-13383 (1998); Prusiner, S. B. Novel proteinaceous infectious particles cause scrapie. Science 216, 136-144 (1982); Kaneko, K. et al. Evidence for protein X binding to a discontinuous epitope on the cellular prion protein during scrapie prion propagation. Proc. Natl. Acad. Sci. USA 94, 10069-10074 (1998); Zulianello, L. et al. Dominant-negative inhibition of prion formation diminished by deletion mutagenesis of the prion protein. J.Virol. 74, 4351-4360 (2000)) The present invention is based in part on determining that antibodies recognizing PrP^{C} prevent the requisite molecular interactions and impede PrP^{Sc} formation. The reported efficiency with which a specific antibody reverses Alzheimer's pathology in vivo is supportive of this viewpoint. (Schenk, D. et al. Immunization with amyloid-beta attenuates Alzheimer-disease-like pathology in the PDAPP mouse. Nature 400, 173-177 (1999); Bard, F. et al. Peripherally administered antibodies against amyloid beta-peptide enter the central nervous system and reduce pathology in a mouse model of Alzheimer disease. Nat. Med. 6, 916-919 (2000)).

### SUMMARY OF THE INVENTION

Molecules and in particular antibodies are disclosed which are characterized by their ability to (a) prevent the formation of infectious proteins (e.g. prions); and/or (b) clear infectious proteins such as prions from infected cells. The molecules such as antibodies and therapeutically active fragments thereof are preferably included in a pharmaceutical formulation which is administered via a method of the invention to treat and/or prevent disease such as prion related diseases which are associated with a particular conformation (e.g. PrP^{Sc}) of a protein (e.g. a PrP protein).

Methodology is disclosed whereby infection with PrP^{Sc} protein is prevented and/or an established PrP^{Sc} infection is treated and PrP^{Sc} in cleared. The method comprising bringing certain molecules into contact with cells and allowing those molecules to remain in contact with the cells for a time and under conditions such that the molecules bind to epitopes on PrP^{C} and thereby hinder a change in conformation from PrP^{C} protein to PrP^{Sc} protein. The molecules are preferably in a pharmaceutical formulation and more preferably comprise Fab fragments selected from the group consisting of D13, D18, R1 and R2.

The present invention focuses on a panel of recombinant antibody Fab fragments, which recognize different epitope regions on PrP, and inhibit prion propagation in PrP^{Sc}-infected cultured mouse neuroblastoma cells (ScN2a). Antibodies disclosed here bind cell surface PrP^{C} and thereby inhibit PrP^{Sc} formation in a dose dependent manner. In cells treated with the most potent antibody, Fab D18, prion replication is completely abolished and pre-existing PrP^{Sc} is rapidly cleared, demonstrating the ability of this antibody to cure an established PrP^{Sc} infection. The potent activity of Fab D18 is associated with (1) its ability to more completely recognize the total population of PrP^{C} molecules on the cell surface relative to other antibodies and (2) with the location of the epitope on a PrP^{C} protein that the Fab D18 binds to. The results provided demonstrate the ability of antibodies to prevent and treat prion diseases and identify a region of PrP^{C} for drug targeting.

The invention further includes an assay device and method of carrying out an assay. The method comprises using high-throughput screening methodology to identify which molecules are capable of hindering binding between an antibody such as D18 and PrP^{C}. The identified molecules could then be tested using methodology described further here to determine their ability to prevent conversion of PrP^{C} protein to PrP^{Sc} protein in the presence of a natural milieu where such a conversion would normally be expected to take place.

An aspect of the invention is molecules including antibodies and fragments thereof which effect the formation and/or clearance of infectious proteins from cells.

There are two further aspects to the invention which are therapeutic in nature. First, antibodies are administered to treat a preexisting prion disease and/or to prevent an animal from developing a prion disease. Second, antigens are administered which generate an immune response (i.e. produce antibodies) and the antibodies generated treat a preexisting prion disease and/or prevent an animal from developing a prion disease.

These and other objects, advantages, and features of the invention will become apparent to those persons skilled in the art upon reading the details of the molecules, antibodies and methodologies as more fully described below.

### BRIEF DESCRIPTIONS OF THE DRAWINGS

FIG. 1 shows the amino acid sequence of mouse PrP protein along with specific differences between mouse PrP and human PrP;

FIG. 2 shows the amino acid sequence of mouse PrP and specifically shows differences between mouse PrP and bovine PrP;

FIG. 3 shows the amino acid sequence of mouse PrP and specifically shows differences between mouse PrP and ovine PrP.

FIG. 4 shows images of Western Blot gels run on the five different antibodies D13, D18, R72, R1 and R2 at different concentrations (in μg/ml) of antibody on an ScN2a cell culture;

FIG. 5 is a graph of the results obtained with four of the five antibodies per FIG. 4 showing the concentration dependent effect;

FIG. 6 shows images of Western Blot gels run on a control of the five antibodies (see FIG. 4) over 1, 2 and 3 weeks with ScN2a cell culture;

FIG. 7 shows an image of Western Blot gels run on a control and the antibodies D13 and D18, R72, R1 and R2 with ScN2a cells over 1, 2, 3 and 4 days;

FIG. 8 is a graph of duration of treatment in days versus the total percent of PrP^{Sc} in the ScN2a cell culture as per FIG. 7;

FIG. 9 is a graph of data which is the mean of three experiments for each of the five antibodies showing the antibody concentration (μg/ml) versus mean fluorescence channel; and

FIG. 10 is three different three dimensional structures for PrP protein.

### DETAILED DESCRIPTION OF THE INVENTION

Before the present molecules, antibodies and methodologies are described, it is to be understood that this invention is not limited to particular molecules, antibodies and methods described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a”, “and”, and “the” include plural referents unless the context clearly dictates otherwise. Thus, for example, reference to “an antibody” includes a plurality of such antibodies and reference to “the molecules” includes reference to one or more molecules and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The terms “treatment”, “treating” and the like are used herein to generally mean obtaining a desired pharmacological and/or physiological effect. The effect may be prophylactic in terms of completely or partially preventing an infection, a disease or symptom thereof and/or may be therapeutic in terms of partially or completely clearing an infectious protein (e.g. prions) curing a disease and/or adverse effect attributed to the disease. The term “treatment” as used herein covers any treatment of a disease in a cell line, a mammal, particularly a human, and includes: (a) preventing the disease from occurring in a subject which may be predisposed to the infection and/or the disease but has not yet been diagnosed as having it; (b) inhibiting the infection or disease, i.e. arresting its development; or (c) relieving the infection or disease, i.e. causing regression of the infection or disease. The present invention is directed towards treating patients with medical conditions relating to infection proteins and in particular prions. Accordingly, a treatment of the invention would involve preventing, inhibiting or relieving any infection or medical condition related to an infectious protein and in particular prions.

As used herein, the term “antibody” (Ab) or “monoclonal antibody” (Mab) is meant to include intact molecules as well as antibody fragments (such as, for example, Fab and F(ab')2 fragments) which are capable of specifically binding to protein. Fab and F(ab')2 fragments lack the Fc fragment of an intact antibody, clear more rapidly from the circulation, and may have less non-specific tissue binding than an intact antibody; see, e.g., Wahl, J. Nucl. Med. 24 (1983), 316-325. Thus, these fragments are preferred, as well as the products of a Fab or other immunoglobulin expression library. Moreover, antibodies of the present invention include chimeric, single chain, humanized antibodies, human antibodies obtainable by or from phage display, a transgenic mouse carrying human immunoglobulin genes and/or human chromosomes, isolated immune cells from human body, in vitro or ex vivo immunization of human immune cells, or any other available methods. Antibodies to PrP proteins are disclosed and described in U.S. Pat. Nos. 5,846,533; 6,290,954 and published PCT application WO 02/10335 and U.S. Pat. No. 4,806,627 disclosing monoclonal antibody 263K 3F4 produced by cell line ATCC HB9222 deposited Oct. 8, 1986 all of which are incorporated herein by reference in their entirety. The cell line producing antibody can be obtained from the American Type Culture Collection 12301 Parklawn Drive, Rockville, Md. 20852.

The terms “prion”, “prion protein”, “PrP^{Sc} protein”, “PrP^{Sc}” and the like are used interchangeably herein to refer to the infectious PrP^{Sc} form of a PrP protein, and is a contraction of the words “protein” and “infection.” Particles are comprised largely, if not exclusively, of PrP^{Sc} molecules encoded by a PrP gene. Prions are distinct from bacteria, viruses and viroids. Known prions infect animals to cause scrapie, a transmissible, degenerative disease of the nervous system of sheep and goats, as well as bovine spongiform encephalopathy (BSE), or “mad cow disease”, and feline spongiform encephalopathy of cats. Four prion diseases known to affect humans are (1) kuru, (2) Creutzfeldt-Jakob Disease (CJD), (3) Gerstmann-Sträussler-Scheinker Disease (GSS), and (4) fatal insomnia (FI). As used herein “prion” includes all forms of prions causing all or any of these diseases or others in any animals used—and in particular in humans and domesticated farm animals.

The terms “conformationally altered protein”, “disease related conformation of a protein” and the like are used interchangeably here to describe any protein which has a three dimensional conformation associated with a disease. The conformationally altered protein may cause the disease, be a factor in a symptom of the disease or appear as a result of other factors associated with the disease. The conformationally altered protein appears in another conformation which has the same amino acid sequence. In general, the conformationally altered protein formed is “constricted” in conformation as compared to the other “relaxed” conformation which is not associated with disease. Those skilled in the art reading this disclosure will recognize the applicability of the antibody formulations of the invention to other conformationally altered proteins even though the invention is described in general as regards to prions. The following is a non-limiting list of diseases with associated proteins which assemble two or more different conformations wherein at least one conformation is an example of a conformationally altered protein.

| Disease | Insoluble Proteins |
|---|---|
| Alzheimer's Disease | APP, Aβ peptide, α1-antichymotrypsin, |
| | tau, non-Aβ component, presenillin 1, |
| | presenillin 2, apoE |
| Prion diseases, Creutzfeldt | PrP^{Sc} |
| Jakob disease, scrapie and | |
| bovine spongiform | |
| encephalopathy | |
| ALS | SOD and neurofilament |
| Pick's disease | Pick body |
| Parkinson's disease | α-synuclein in Lewy bodies |
| Frontotemporal dementia | tau in fibrils |
| Diabetes Type II | Amylin |
| Multiple myeloma- | IgGL-chain |
| Plasma cell dyscrasias | |
| Familial amyloidotic | Transthyretin |
| polyneuropathy | |
| Medullary carcinoma of thyroid | Procalcitonin |
| Chronic renal failure | β₂-microglobulin |
| Congestive heart failure | Atrial natriuretic factor |
| Senile cardiac and systemic | Transthyretin |
| amyloidosis | |
| Chronic inflammation | Serum Amyloid A |
| Atherosclerosis | ApoAl |
| Familial amyloidosis | Gelsolin |
| Huntington's disease | Huntington |

The terms “sterilizing”, “making sterile” and the like are used here to mean rendering something non-infectious or rendering something incapable of causing a disease. Specifically, refers to rendering a protein non-infectious or incapable of causing a disease or the symptoms of a disease. Still more specifically, it refers to rendering a conformationally altered protein (e.g., PrP^{Sc} known as prions) incapable of causing a disease or the symptoms of a disease.

By “effective dose” or “amount effective” is meant an amount of a compound sufficient to provide the desired sterilizing result. This will vary depending on factors such as (1) the active agent used, (2) the combination of antibodies in the formulation, (3) the type of object or material being sterilized, and (4) the amount or concentration of infectious proteins which might be present. Formulations of the invention or more specifically antibody formulations of the invention could be mixed with a material in an amount in a range 1 to 500 μg of antibody per ml or mg of material being sterilized. The concentration is sufficient if the resulting composition is effective in decreasing (preferably eliminating) the infectivity of conformationally altered proteins such that the treated material over time would not result in infection. Because (1) some materials will have higher concentrations of altered protein than others (2) some materials are contacted more frequently than others and (3) individual proteins have different degrees of infectivity the effective dose or concentration range needed to sterilize can vary considerably. It is also pointed out that the dose needed to treat an amount of material may vary somewhat based on the pH the treatment is carried out at and the amount of time the compound is maintained in contact with the material at the desired low pH (e.g., 4.5 or less) level and the surrounding temperature and pressure.

The term “LD₅₀” as used herein is the dose of an active substance that will result in 50 percent lethality in all treated experimental animals. Although this usually refers to invasive administration, such as oral, parenteral, and the like, it may also apply to toxicity using less invasive methods of administration, such as topical applications of the active substance e.g. antibody containing formulation.

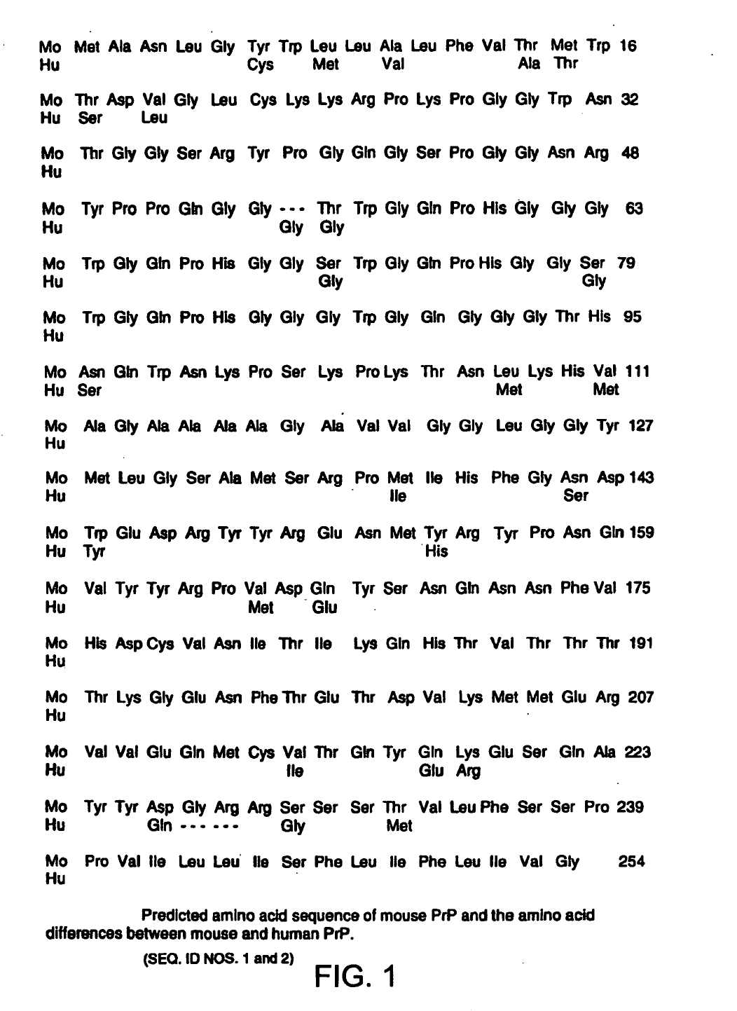

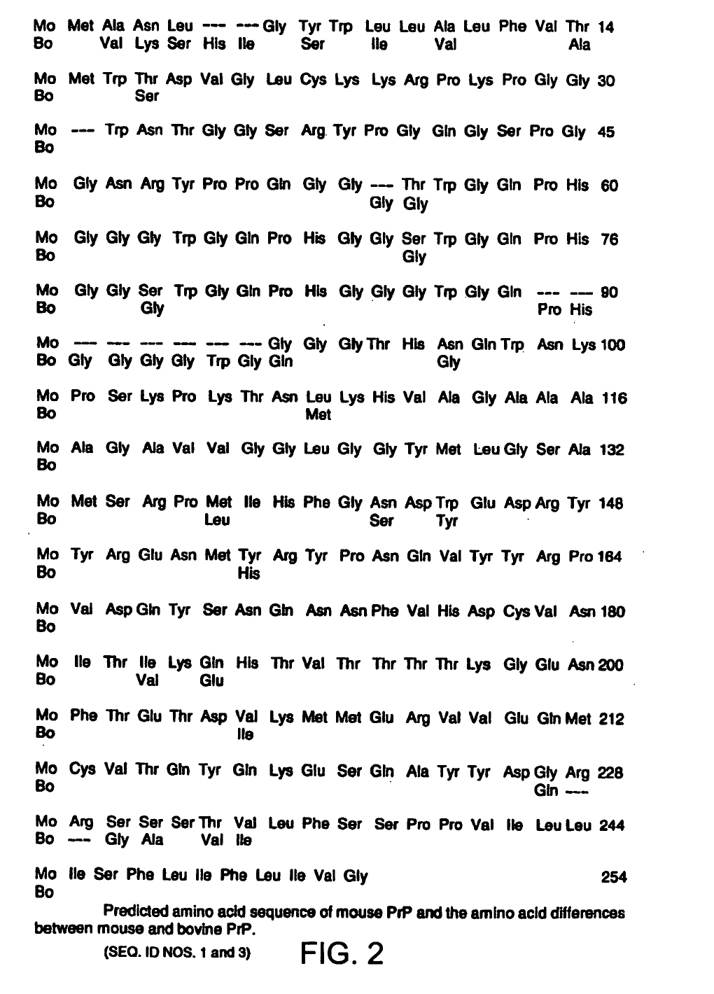

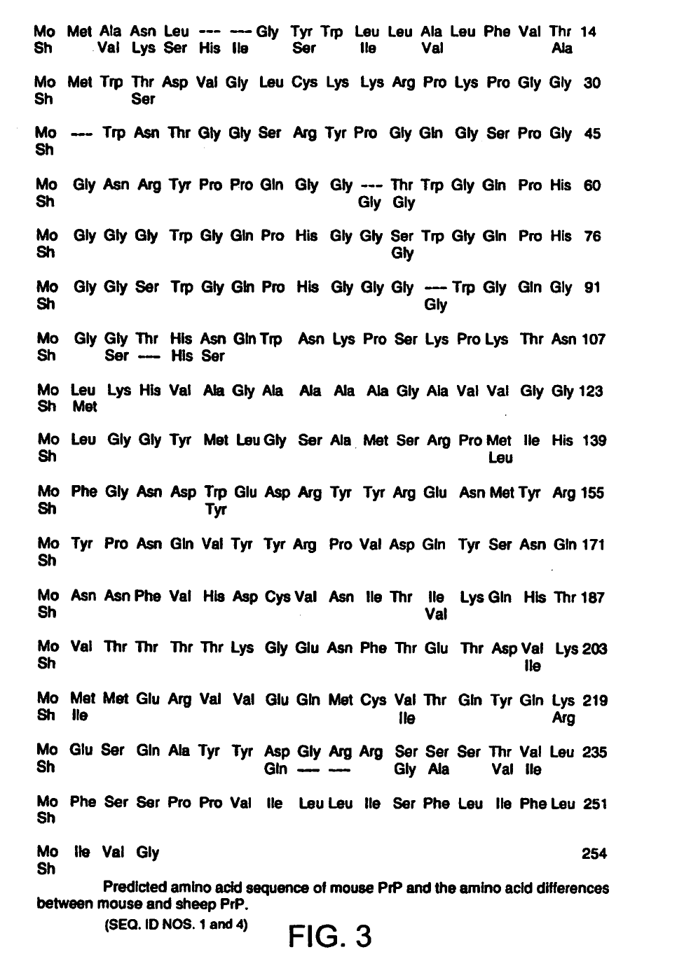

The term “PrP gene” is used herein to describe genetic material which expresses proteins including known polymorphisms and pathogenic mutations. The term “PrP gene” refers generally to any gene of any species which encodes any form of a prion protein. Some commonly known PrP sequences are described in Gabriel et al., Proc. Natl. Acad. Sci. USA 89:9097-9101 (1992) and U.S. Pat. Nos. 5,565,186 and 5,792,901, incorporated herein by reference to disclose and describe such sequences and see attached sequences of FIG. 1-3. The PrP gene can be from any animal, and any and all polymorphisms and mutations thereof, it being recognized that the terms include other such PrP genes that are yet to be discovered. The protein expressed by such a gene can assume either a PrP^{c} (non-disease) or PrP^{Sc} (disease) form.

The terms “standardized prion preparation”, “prion preparation”, “preparation” and the like are used interchangeably herein to describe a composition (e.g., brain homogenate) obtained from the brain tissue of mammals which exhibits signs of prion disease: the mammal may (1) include a transgene as described herein; (2) have and ablated endogenous prion protein gene; (3) have a high number of prion protein gene from a genetically diverse species; and/or (4) be a hybrid with an ablated endogenous prion protein gene and a prion protein gene from a genetically diverse species. Different combinations of 1-4 are possible, e.g. 1 and 2. The mammals from which standardized prion preparations are obtained exhibit clinical signs of CNS dysfunction as a result of inoculation with prions and/or due to developing the disease of their genetically modified make up, e.g., high copy number of prion protein genes. Standardized prion preparations and methods of making such are described and disclosed in U.S. Pat. No. 5,908,969 issued Jun. 1, 1999 and U.S. Pat. No. 6,020,537 issued Feb. 1, 2000 both of which are incorporated herein by reference in their entirety to disclose and describe standardized prion preparations.

The term “Alzheimer's disease” (abbreviated herein as “AD”) as used herein refers to a condition associated with formation of neuritic plaques comprising amyloid β protein, primarily in the hippocampus and cerebral cortex, as well as impairment in both learning and memory. “AD” as used herein is meant to encompass both AD as well as AD-type pathologies.

The term “AD-type pathology” as used herein refers to a combination of CNS alterations including, but not limited to, formation of neuritic plaques containing amyloid β protein in the hippocampus and cerebral cortex. Such AD-type pathologies can include, but are not necessarily limited to, disorders associated with aberrant expression and/or deposition of APP, overexpression of APP, expression of aberrant APP gene products, and other phenomena associated with AD. Exemplary AD-type pathologies include, but are not necessarily limited to, AD-type pathologies associated with Down's syndrome that is associated with overexpression of APP.

The term “phenomenon associated with Alzheimer's disease” as used herein refers to a structural, molecular, or functional event associated with AD, particularly such an event that is readily assessable in an animal model. Such events include, but are not limited to, amyloid deposition, neuropathological developments, learning and memory deficits, and other AD-associated characteristics.

The term “cerebral amyloid angiopathy” (abbreviated herein as CAA) as used herein refers to a condition associated with formation of amyloid deposition within cerebral vessels which can be complicated by cerebral parenchymal hemorrhage. CAA is also associated with increased risk of stroke as well as development of cerebellar and subarachnoid hemorrhages (Winters (1987) Stroke 18:311-324; Haan et al. (1994) Dementia 5:210-213; Itoh et al. (1993) J. Neural. Sci. 116:135-414). CAA can also be associated with dementia prior to onset of hemorrhages. The vascular amyloid deposits associated with CAA can exist in the absence of AD, but are more frequently associated with AD.

The term “phenomenon associated with cerebral amyloid angiopathy” as used herein refers to a molecular, structural, or functional event associated with CAA, particularly such an event that is readily assessable in an animal model. Such events include, but are not limited to, amyloid deposition, cerebral parenchymal hemorrhage, and other CAA-associated characteristics.

The term “β-amyloid deposit” as used herein refers to a deposit in the brain composed of Aβ as well as other substances.

Abbreviations used herein include:
CNS for central nervous system;
BSE for bovine spongiform encephalopathy;
CJD for Creutzfeldt-Jakob Disease;
FFI for fatal familial insomnia;
GSS for Gerstmann-Sträussler-Scheinker Disease;
AD for Alzheimer's disease;
CAA for cerebral amyloid angiopathy;
Hu for human;
HuPrP for human prion protein;
Mo for mouse;
MoPrP for mouse prion protein;
SHa for a Syrian hamster;
SHaPrP for a Syrian hamster prion protein;
PAMAM for polyamidoamide dendrimers;
PEI for polyethyleneimine;
PK for proteinase K
PPI for polypropyleneimine;
PrP^{Sc} for the scrapie isoform of the prion protein;
PrP^{C} for the cellular contained common, normal isoform of the prion protein;
PrP 27-30 or PrP^{Sc} 27-30 for the treatment or protease resistant form of PrP^{Sc};
MoPrP^{Sc} for the scrapie isoform of the mouse prion protein;
N2a for an established neuroblastoma cell line used in the present studies;
ScN2a for a chronically scrapie-infected neuroblastoma cell line;
ALS for amyotrophic lateral sclerosis;
HD for Huntington's disease;
FTD for frontotemporal dementia;
SDS for sodium dodecyl sulfate;
SOD for superoxide dismutase.

### INVENTION IN GENERAL

The invention is generally directed toward preventing a non-pathogenic form of a protein from converting to a pathogenic form of a protein. In a preferred embodiment prP^{C} (which is non-pathogenic) is prevented from being converted to PrP^{Sc} (which is pathogenic) when PrP^{C} is in the presence of a surrounding environment of its natural milieu where conversion of PrP^{C} to PrP^{Sc} would normally be expected. The conversion of the non-pathogenic form of a protein to a pathogenic form is generally prevented by binding a compound (such as an antibody) to the non-pathogenic form of the protein.

While not being bound to any thereby regarding the mechanism of action, it is believed that by binding a compound (such as an antibody) to the non-pathogenic protein (such as a PrP^{C} protein) the non-pathogenic form of the protein is rendered incapable of interacting with and/or binding to another molecule or molecules necessary to bring about the conversion to the pathogenic form of the protein (such as PrP^{Sc}). For example, D18 binds to PrP^{C} and prevents PrP^{C} from interacting with PrP^{Sc} and thereby prevents the conversion of the PrP^{C} to PrP^{Sc}. By preventing the formation of pathogenic protein such as PrP^{Sc} the cells may (on their own or with the aid of the binding antibody) clear PrP^{Sc} from the system.

Molecules such as antibodies which bind PrP^{C} may be used alone or in any desired combination. Further, the binding molecules or antibodies may be designed to bind to PrP^{C} from different mammals (e.g. cow, human, sheep, mouse, etc.) and different PrP variants.

The invention encompasses an assay used to find compounds of interest. The assay involves screening compounds and in particular screening libraries of organic compounds to determine which compounds block known binding reactions such as that between PrP^{C} and D18. The compounds identified in the assay are potentially useful as both therapeutics to treat prion diseases and disinfectants to render prions non-infectious and non-pathogenic. Thus, pharmaceutical formations can be prepared by combining the compounds with a pharmaceutically acceptable carrier and other compositions can be prepared for disinfecting by combining the compounds with other compositions such as soaps including surgical soaps which may be further comprised of antibacterial and antiviral agents.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Fab Fragments Block PrP^{C} to PrP^{Sc} Conversion. Antibody inhibition of prion propagation was investigated using recombinant PrP-specific Fab fragments D13, D18, R1, R2 and R72 (Williamson, R. A. et al. Circumventing tolerance in order to generate autologous monoclonal antibodies to the prion protein. Proc. Natl. Acad. Sci. USA 93, 7279-7282 (1996); Peretz, D. et al. A conformational transition at the N-terminus of the prion protein features in formation of the scrapie isoform. J. Mol. Biol. 273, 614-622 (1997); Williamson, R. A. et al. Mapping the prion protein using recombinant antibodies. J. Virol. 72, 9413-9418 (1998)) the binding epitopes of which are shown below in Table 1.

### Table 1

Epitopes recognized and dissociation constants for the binding of PrP-specific Fabs to recombinant PrP(29-231) refolded into an α-helical conformation. Binding constants were determined by surface plasmon resonance. The numbering of the residues throughout and in particular in the “Epitope” column of Table 1 is numbering which corresponds to residue numbering of mouse PrP as shown in FIG. 1.

| Fab | Epitope | Kd (μg/ml) | Kd (nM) |
|---|---|---|---|
| D13 | 95-103 | 0.18 ± 0.03 | 3.5 ± 0.5 |
| D18 | 132-156 | 0.07 ± 0.03 | 1.5 ± 0.6 |
| R72 | 151-162 | no binding | no binding |
| R1 | 220-231 | 0.09 ± 0.04 | 1.8 ± 0.8 |
| R2 | 220-231 | 0.11 ± 0.02 | 2.2 ± 0.5 |

As determined by surface plasmon resonance (SPR), the antibodies bind tightly to recombinant α-helical PrP(29-231) (Table 1). Fab R72 does not recognize PrP in SPR or on the cell surface, but does bind to PrP^{C} coated onto the surface of ELISA wells ( Peretz, D. et al. A conformational transition at the N-terminus of the prion protein features in formation of the scrapie isoform. J. Mol. Biol. 273, 614-622 (1997)).

A range of concentrations (0.3 to 20 μg/ml) of each antibody was added to ScN2a cultures for a period of seven days. After seven days, cells were harvested and the level of PrP^{Sc} in the culture analyzed by immunoblotting.

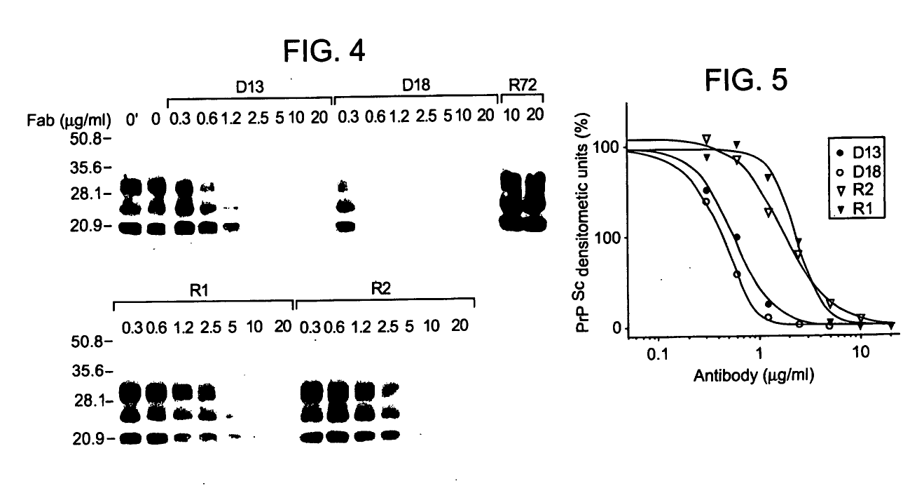

The gel images shown in FIG. 4 show that with the exception of those cells treated with Fab R72, the level of PrP^{Sc} in the antibody-treated cells (as compared with non-treated cells) was dramatically reduced in a dose-dependent manner. These results are plotted in the graph of FIG. 5.

By this analysis, Fabs D13 and D18 appear to be approximately equally effective, having IC₅₀ values of 0.45 μg/ml (9 nM) and 0.6 μg/ml (12 nM), respectively. Fabs R1 and R2 were slightly less efficient, with IC₅₀ values of 2.5 μg/ml (50 nM) and 2.5 μg/ml (40 nM), respectively. Treatment with Fab R72 did not reduce the level of PrP^{Sc} in the ScN2a culture even when the antibody was used at a concentration of 20 μg/ml and such is clearly shown in FIG. 4.

Dose-dependent inhibition of PrP^{Sc} formation in ScN2a cell culture by PrP-specific recombinant antibody Fabs is shown via the gel images of FIG. 4. PrP^{Sc} levels in ScN2a cells were measured by immunoblotting following 7 days of culture in the presence of antibodies D13, D18, R72, R1 or R2 at concentrations of 0-20 μg/ml. Lane 0' of FIG. 4 indicates the level of PrP^{Sc} in the ScN2a culture prior to antibody treatment. Densitometric measurement of PrP^{Sc} bands identified in the immunoblot given in FIG. 4. Values are given as absolute densitometric units (DU), where 100% is equivalent to the intensity of the PrP^{Sc} band in the absence of antibody treatment and 0% denotes undetectable levels of PrP^{Sc} in the culture (no band). Data plotted in FIG. 5 represent the mean from three independent experiments.

During these experiments the levels of PrP^{C} and glyceraldehyde-3-phosphate dehydrogenase in antibody-treated and untreated cells were found to be invariant, indicating that the PrP-specific antibodies used produced no cytotoxic effects that may have indirectly compromised the production of PrP^{Sc} .

### Example 2

Fab Fragments Render Cells Immune. Because the results of EXAMPLE 1 showed that it was possible to dramatically reduce PrP^{Sc} levels in ScN2a cells, the studies were extended to determine whether PrP^{Sc} could be maintained at undetectable levels even after the removal of PrP-specific antibody from the experimental system. ScN2a cells were independently passaged for a minimum of 7 days in the presence of 10 μg/ml of each of the recombinant Fabs.

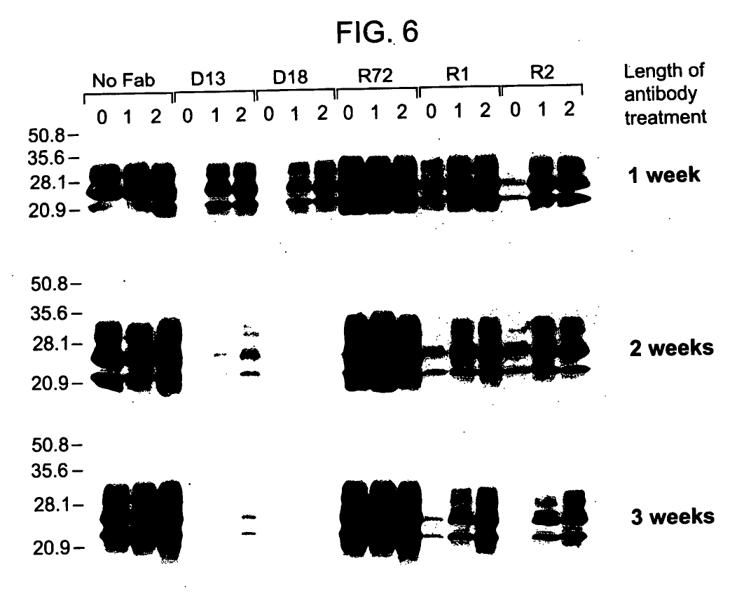

Antibody was then removed from the culture and the cells passaged for an additional period in Fab-free medium, after which time the level of PrP^{Sc} was re-measured. The results are show in the gel images of FIG. 6.

Elimination of PrP^{Sc} from prion infected cells over time is shown in FIG. 6. ScN2a cells were cultured for 1, 2 or 3 weeks in the presence of 10 μg/ml of Fabs D13, D18, R72, R1 or R2, or in the absence of antibody. The level of PrP^{Sc} in the culture was then analyzed by immunoblot either immediately after the termination of antibody treatment (lane 0) of FIG. 6 or after cells were passaged for an additional 1 (lane 1) or 2 (lane 2) weeks in the absence of antibody.

FIG. 6 shows that PrP^{Sc} concentrations in ScN2a cells passaged for 1 week in the presence of Fab D18 were reduced to non-detectable levels, but returned to approximately 50% of the level of an untreated control culture after 1 additional week of growth in the absence of D18. However, if cells were cultured for a 2-week period in the presence of Fab D18 then PrP^{Sc} remained at undetectable levels after 4 additional weeks of culture in antibody-free medium. Similarly, when prion infected cells were treated with Fab D13 for 3 consecutive weeks followed by 1 week of growth in media without antibody, no PrP^{Sc} could be detected, although after an additional week in culture without Fab, PrP^{Sc} increased back to 5% of the level found in untreated control culture. If, however, cells were subjected to 4 consecutive weeks of treatment with Fab D13, PrP^{Sc} remained below the level of detection after 4 weeks of culture without antibody. Because nothing was detected the 4 week results are not shown in FIG. 6.

When employed at a concentration of 10 μg/ml neither Fab R1, nor Fab R2, was sufficiently potent to prevent the re-emergence of PrP^{Sc} in the culture after antibody was removed. Fab R72 had no impact on the level of PrP^{Sc} in the culture after three, or nine consecutive weeks of treatment.

### Example 3

Effects of Fab Fragments on Mice. As a second measure of prion titer, bioassays were carried out in which CD-1 Swiss mice were inoculated with antibody-treated (10 μg/ml) and untreated ScN2a cells. Mice inoculated intracerebrally with D18, D13, or R2-treated cells were disease free after a period of 230 days, whereas mice inoculated with untreated or R72-treated cells had a mean incubation time to disease of 169 and 165 days, respectively. The prolonged incubation times correspond to a reduction of over 3 logs in the infectious prion titer in treated cells (Butler, D. A. et al. Scrapie-infected murine neuroblastoma cells produce protease-resistant prion proteins. J. Virol. 62, 1558-1564 (1988)).

### Example 4

Clearing Preexisting PrP^{Sc}. The results of the above Examples do not show how efficiently PrP^{Sc} preexisting in the ScN2a cultures at the onset of antibody treatment is subsequently eliminated from the cells. The above data might be taken to indicate that PrP^{Sc} levels rapidly diminish. Indeed, data reported in other studies describing the inhibition of prion propagation have been interpreted in this way (See Caughey, B. and Raymond, G. J. Sulfated polyanion inhibition of scrapie-associated PrP accumulation in cultured cells. J. Virology 67, 643-650 (1993); Chabry, J. et al. Species-independent inhibition of abnormal prion protein (PrP) formation by a peptide containing a conserved PrP sequence. J. Virol. 73, 6245-6250 (1999); Perrier, V. et al. Mimicking dominant negative inhibition of prion replication through structure-based drug design. Proc. Natl. Acad. Sci. USA 97, 6073-6078 (2000); Caughey, W. S., Raymond, L. D., Horiuchi, M., and Caughey, B. Inhibition of protease-resistant prion protein formation by porphyrins and phthalocyanines. Proc. Natl. Acad. Sci. USA 95, 12117-12122 (1998); Supattapone, S., Nguyen, H. O., Cohen, F. E., Prusiner, S. B., and Scott, M. R. Elimination of prions by branched polyamines and implications for therapeutics. Proc. Natl. Acad. Sci. USA 96, 14529-14534 (1999)). However, such an interpretation fails to take into consideration the dynamic expansion of ScN2a populations in culture. Critically, if an antibody or other reagent curtails the formation of nascent PrP^{Sc} molecules, then each successive round of cell division may serve to dilute the effective concentration, but not necessarily the total amount, of residual PrP^{Sc} within the culture, thus creating a potentially erroneous impression of PrP^{Sc} clearance. An accurate calibration of the rate with which PrP^{Sc} is purged from ScN2a cultures must therefore account for any increase in cell population and commensurate reduction in PrP^{Sc} concentration that has taken place over the course of the experiment.

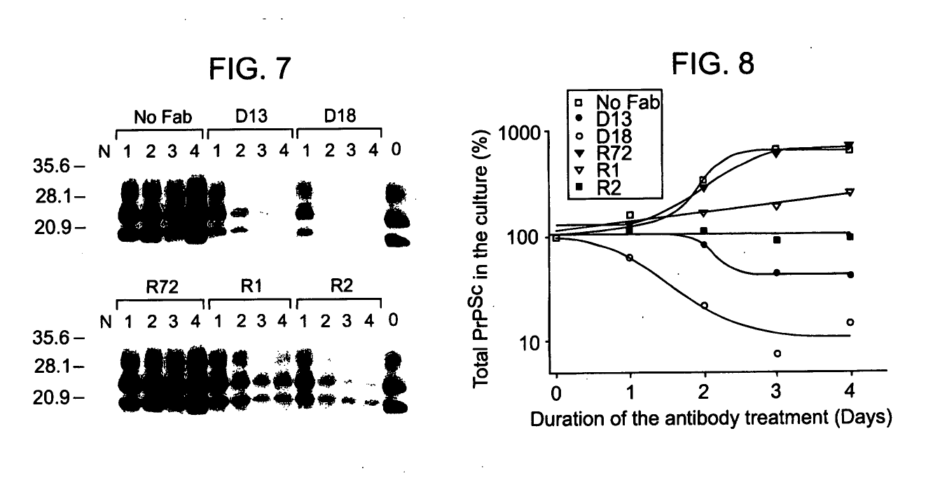

To more thoroughly analyze the kinetics of prion clearance in this experimental system, ScN2a cells were independently grown in the presence of 10 μg/ml of each PrP-specific Fab. Cells were harvested after a period of 1, 2, 3, and 4 days of antibody treatment and the total mass of cell protein determined in each case as a measure of cell number. The PrP^{Sc} concentrations in Fab-treated and untreated cells at these time points were determined by immunoblotting as shown in FIG. 7. Total PrP^{Sc} in the culture at each time point was then calculated by factoring in the total cell mass in each case. When these data were plotted against the duration of antibody treatment, more meaningful differences in the efficacy with which individual Fabs resolved prion infection became apparent—see FIG. 8.

The gel images of FIG. 7 show the time course of antibody-mediated PrP^{Sc} clearance. The level of PrP^{Sc} in ScN2a cells grown for 1, 2, 3 or 4 days in the presence of PrP-specific Fabs (10 μg/ml) was determined by immunoblotting. The effect of antibody treatment on the total amount of PrP^{Sc} in ScN2a cell cultures. The data represent the mean of three experiments and is plotted on the graph of FIG. 8.

This Example is similar to the results of the other Examples in showing Fab D18 was found to be the most effective antibody. The time taken from the initial treatment with D18 to eliminate 50% of PrP^{Sc} from the cells (t_{1/2}) was 28 h. The t_{1/2} of PrP^{Sc} in ScN2a cells is thought to exceed 24 h (Borchelt, D. R., Scott, M., Taraboulos, A., Stahl, N., and Prusiner, S. B. Scrapie and cellular prion proteins differ in their kinetics of synthesis and topology in cultured cells. J. Cell Biol. 110, 743-752 (1990)), suggesting that at a concentration of 10 μg/ml, Fab D18 is able to completely abolish prion propagation and that preexisting PrP^{Sc} is subsequently eliminated from the cells. This finding indicates that at least a certain amount of PrP^{Sc} is continuously expunged from ScN2a cultures through normal degradation pathways. Fab D13 was the next most potent antibody, also lowering the level of PrP^{Sc} in the culture, but to a lesser extent than in D18 treated cells, indicating that in the presence of this Fab, there may be a minimal level of residual PrP^{Sc} synthesis. Fabs R1 and R2, although clearly reducing the rate of prion propagation in ScN2a cells, are not sufficiently effective to yield a reduction in the overall quantity of PrP^{Sc} present in the culture. In untreated cultures, or cultures treated with Fab R72, prion propagation remained unaffected, and PrP^{Sc} levels increased in tandem with growth in ScN2a cell population.

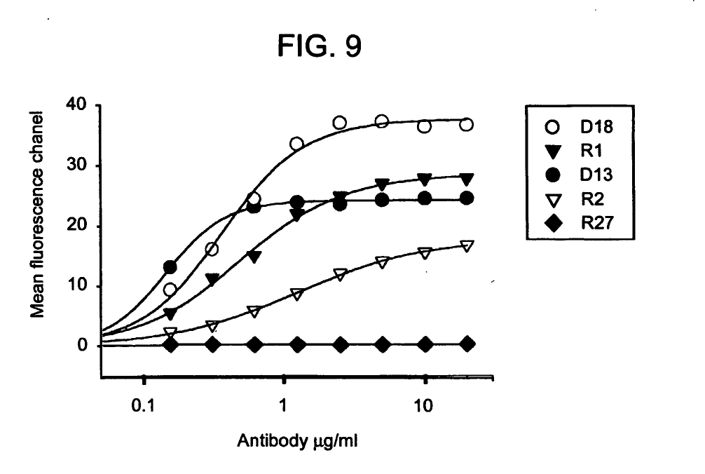

Results provided here clearly show not only that certain molecules such as D18 can potently inhibit prion replication in infected cell cultures, but also that the efficiency of this process varies dramatically between individual antibodies. Mechanistically, the inhibitory effect is most readily explained by antibody binding specifically to PrP^{C} molecules on the cell surface and thereby hindering docking of PrP^{Sc} template or other co-factor critical for the conversion of PrP^{C} to PrP^{Sc}. In good agreement with this hypothesis, Fab D18, which was by far the most effective antibody evaluated here, was distinguished by its capacity to bind a significantly greater number of cell surface PrP^{C} molecules than Fabs D13, R1 or R2 (see FIG. 9).

The results plotted in FIG. 9 show PrP-specific antibody Fab binding to the surface of ScN2a cells. The reactivity of PrP-specific Fabs D13, D18, R72, R1 and R2 with the surface of ScN2a cells was evaluated using flow cytometry. The data plotted in FIG. 9 represent the mean of three experiments.

In contrast, Fab R72, which had no effect on prion propagation, failed to recognize cell surface PrP^{C} even at a concentration of 20 μg/ml. These results show that the fraction of total cell surface PrP^{C} occupied by a given antibody is a key determinant of the inhibitory potency of that antibody. In addition, the data presented in FIG. 9 suggest that discrete PrP^{C} populations, distinguished on the basis of antibody reactivity, exist on the surface of ScN2a cells. These populations may be conformationally distinct from one another, may differ in their post-translational modification, or may result from the interaction of a proportion of PrP^{C} with other cell surface components, possibly including other molecules of PrP.

### Example 5

PrP Binding Region Effecting PrP^{C} to PrP^{Sc} Conversion. Additional work was carried out to show whether the region of PrP bound by each antibody was of intrinsic importance to its inhibitory potency. To address this question we looked at conditions in which equal amounts of two different Fabs were bound to the ScN2a cell surface. For example, when used at concentrations of 0.6 μg/ml equivalent amounts of Fabs D18 and D13 were bound to the cell surface (FIG. 9), but D18 inhibited prion replication much more efficiently (FIG. 4). Similarly, at concentrations of 0.6 μg/ml and 2.5 μg/ml respectively, Fabs D18 and R1 bound equivalently to ScN2a cells, but D18 was clearly more effective in reducing the level of PrP^{Sc} in the culture. Finally, at a concentration of 2.5 μg/ml Fabs D13 and R1 bound equivalently to the cell surface, but D13 more actively reduced PrP^{Sc} synthesis.

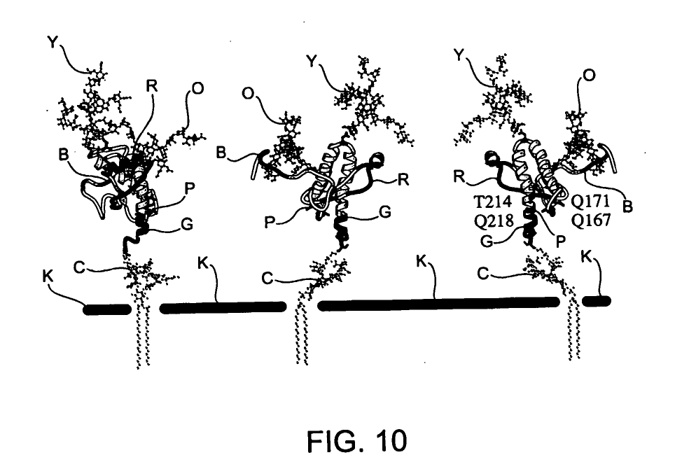

This analysis demonstrates that the region of PrP^{C} bound by a given antibody is a critical determinant of its inhibitory capacity, and therefore provides new insight into the specific machinery of prion propagation. The known binding epitope of Fab D18 spans PrP residues 132-156 (Williamson, R. A. et al. Mapping the prion protein using recombinant antibodies. J. Virol. 72, 9413-9418 (1998).) and incorporates helix A (residues 145-155) of PrP^{C} —see FIGS. 1-3 and 3-D structures of FIG. 10.

The structures of FIG. 10 show the regions of sequence recognized by Fabs D13 (blue, represented by “B”), D18 (red, represented by “R”) and R1/R2 (green, represented by “G”) superimposed onto three views of the NMR structure of recombinant PrP(90-231) (James, T. L. et al. Solution structure of a 142-residue recombinant prion protein corresponding to the infectious fragment of the Scrapie isoform. Proc. Natl. Acad. Sci. USA 94, 10086-10091 (1997)). Carbohydrate moieties linked to Asn 180 and Asn 196 are shown in orange (represented by “O”) and yellow (represented by “Y”), respectively (Rudd, P. M. et al. Glycosylation differences between the normal and pathogenic prion protein isoforms. Proc. Natl Acad. Sci. USA 96, 13044-13049 (1999)). The COOH-terminal GPI-anchor is shown in cyan (represented by “C”) extending into the cell membrane (black, represented by “K”). Side chains of residues Q167, Q171, T214 and Q218, which are proposed to bind to a cellular cofactor critical to prion propagation, are included (purple, represented by “P”).

Spatially, the sequence of residues 145-155 is positioned on the opposite face of the protein from residues Q167, Q171, T214 and Q218, which are hypothesized to participate in binding an auxiliary molecule essential to prion propagation (Kaneko, K. et al. Evidence for protein X binding to a discontinuous epitope on the cellular prion protein during scrapie prion propagation. Proc. Natl. Acad. Sci. USA 94, 10069-10074 (1998); Zulianelloi L. et al. Dominant-negative inhibition of prion formation diminished by deletion mutagenesis of the prion protein. J. Virol. 74, 4351-4360 (2000)). This shows that D18 operates mechanistically by directly blocking or modifying PrP^{C} interaction with PrP^{Sc}, rather than by inhibiting the binding of a cofactor. A number of other reports also identify the 132-156 region of the protein as critical to prion synthesis and inter-species transmission (Scott, M. et al. Propagation of prions with artificial properties in transgenic mice expressing chimeric PrP genes. Cell 73, 979-988 (1993); Priola, S. A. and Chesebro, B. A single hamster PrP amino acid blocks conversion to protease-resistant PrP in scrapie-infected mouse neuroblastoma cells. J. Virology 69, 7754-7758 (1995); Kocisko, D. A. et al. Species specificity in the cell-free conversion of prion protein to protease-resistant forms: a model for the scrapie species barrier. Proc. Natl. Acad. Sci. USA 92, 3923-3927 (1995).), although prion propagation can proceed in the absence of sequence between residues 140 and 175 (Supattapone, S. et al. Prion protein of 106 residues creates an artificial transmission barrier for prion replication in transgenic mice. Cell 96, 869-878 (1999)), indicating that helix A of PrP^{C} is not prerequisite for PrP^{Sc} binding. Based on results obtained here the 132-140 portion of PrP^{C} is shown to be a target for anti-prion drug development.

In contrast to D18, Fabs R1 and R2 are relatively poor inhibitors of prion propagation. Both these antibodies bind the extreme C-terminus of PrP, much closer to the Q167, Q171, T214 and Q218 cluster and distant from the D18 epitope—see FIG. 10. These antibodies may have little effect upon PrP^{Sc} binding, but could be in direct competition with the putative cellular cofactor for binding to PrP^{C} . The lower cell surface binding we observe for Fabs R1 and R2 compared to D8 (FIG. 9) may be explained in part by this type of competition, but the proximity of this epitope to the GPI-anchor and cell surface may also be of importance in this respect.

Little insight into the mechanism of PrP^{Sc}-PrP^{C} interactions can be discerned from the inhibitory activity of Fab D13, since its epitope (residues 95-103) is found in the unstructured portion of the protein. This portion of PrP^{C} is thought to undergo substantial conformational rearrangement as prion infectivity is acquired (Peretz, D. et al. A conformational transition at the N-terminus of the prion protein features in formation of the scrapie isoform. J. Mol. Biol. 273, 614-622 (1997); Leclerc, E., Peretz, D., Ball, H., Sakurai, H., Legname, G., Serban, A., Prusiner, S. B., Burton, D. R., and Williamson, R. A. Immobilized prion protein undergoes spontaneous rearrangement to a conformation having features in common with the infectious form. The inhibitory potency of D13 may derive from its ability to preserve PrP^{C}-like conformation in this segment of the protein. Significantly, with the exception of Fabs R1 and R2, none of the antibodies described here compete with each other for binding to cell surface PrP^{C}. Accordingly, the antibodies may be used therapeutically in combination to achieve maximum inhibitory effect.

The results provided here show that antibodies binding to defined regions of PrP^{C} will efficiently inhibit prion replication. For in vivo applications, Fab fragments have the disadvantage of a short half-life, and may not efficiently traverse from the peripheral circulation into the CNS. Whole antibody molecules prepared from the Fab fragments will likely be more useful, but may require engineering to prevent the recruitment of immunologic effector mechanisms to antibody coated cells (Xu, D. et al. In vitro characterization of five humanized OKT3 effector function variant antibodies. Cell. Immunol. 200, 16-26 (2000); Idusogie, E. E. et al. Mapping of the Clq binding site on rituxan, a chimeric antibody with a human IgG1 Fc. J. Immunol. 164, 4178-4184 (2000)). The above studies, in combination with recent advances targeting Alzheimer's disease (Schenk, D. et al. Immunization with amyloid-beta attenuates like-disease-like pathology in the PDAPP mouse. Nature 400, 173-177 (1999); Bard, F. et al. Peripherally administered antibodies against amyloid beta-peptide enter the central nervous system and reduce pathology in a mouse model of like disease. Nat. Med. 6, 916-919 (2000)), indicate that specific antibodies may become a powerful weapon in the fight against neurodegenerative diseases associated with the accumulation of misfolded proteins.

### Methods and Materials

Details regarding various methods and materials used in the Examples are provided below.

Expression and purification of recombinant PrP-specific antibodies. E.coli 33B6 transformed with plasmid encoding PrP-specific Fab were fermented for 48 h using a Biostat® B controller (B.Braun, Germany) and 1 L media containing MT-8 salts (0.26 g potassium phosphate dibasic, 0.13 g sodium phosphate monobasic dihydrate, 0.5 g ammonium sulphate, 0.1 g sodium citrate dihydrate and 0.15 g potassium chloride per liter), 0.5 g isoleucine, 20% NZ amines, 20% yeast extract, 1 mM magnesium sulphate, 50% glucose, trace metals and 100 μg/ml of ampicillin. Bacterial paste was resuspended in 5 volumes of 2 mM imidazole, 20 mM sodium phosphate, 250 mM sodium chloride, pH 7.0, and processed twice in a Microfluidizer® M-110 EH (Microfluidics Co., USA). The processed paste was titrated to 0.1% PEI (5% stock solution, pH 8.0) and stirred at 4° C. for 30 min, then centrifuged at 10,000 rpm for 30 min at 4° C. The supernatant was diluted in an equal volume of 20 mM imidazole pH 7.0, and loaded onto a SP-Fast Flow Sepharose (Amershamn Pharmacia, Sweden) column. Recombinant Fab was eluted with a linear gradient of 0 to 100% of 20 mM imidazole, 500 mM sodium acetate, pH 7.0, then directly applied to an IMAC column. Antibody was eluted from this column with 200 mM imidazole pH 7.0, then thoroughly dialyzed at 4° C. against 10 mM Tris-HCl, pH 7.2. The dialyzed samples were further purified by elution from a QS-Fast Flow Sepharose column utilizing a linear gradient of 0 to 100% of 10 mM Tris-HCl, 500 mM sodium chloride, pH 7.2 and sterilized by filtration.

Cell culture. Mouse neuroblastoma cells (N2a) were obtained from the American Type Culture Collection. Prion-infected mouse neuroblastoma (ScN2a) cells have been described previously (Butler, D. A. et al. Scrapie-infected murine neuroblastoma cells produce protease-resistant prion proteins. J. Virol. 62, 1558-1564 (1988); Race, R. E., Fadness, L. H., and Chesebro, B. Characterization of scrapie infection in mouse neuroblastoma cells. J. Gen. Virol. 68, 1391-1399. 87). Stock cultures of ScN2a and N2a cells were maintained in MEM, 10% Fetal Bovine Serum (FBS), 2 mM Glutamax (GIBCO BRL), 100 units/ml penicillin and 100 μg/ml streptomycin sulfate in a humidified 37° C. incubator with 5% CO₂. Cells were split 1:15 weekly using 0.05% (w/v) Trypsin-EDTA (GIBCO BRL).

Antibody inhibition studies. PrP-specific antibodies were added to 2×10⁵ ScN2a cells and incubated for an appropriate time according to individual experimental protocols. The cells were fed three times a week with replacement media containing the appropriate amount of antibody. In inhibition experiments in which ScN2a cultures required splitting, cells were detached from culture plates using cell dissociation buffer (GIBCO BRL), rather than with trypsin, because enzymatic activity may have modulated the level of PrP^{Sc}. Cells were harvested in situ by washing 3 times with calcium- and magnesium-free PBS and resuspension in 1 ml lysis buffer (10 mM Tris pH 7.5, containing 150 mM NaCl, 0.5% sodium deoxycholate, 0.5% nonident P-40). Cell nuclei were removed from the lysate by centrifugation at 2,000× g for 2 min, and the protein concentration of the supernatant measured by bicinchoninic acid assay (BCA, Pierce).

Quantitation of PrP^{Sc} by Western blotting and densitometry. ScN2a cell lysates (1 mg/ml) were treated with 20 μg/ml of proteinase K (total protein/enzyme ratio, 50:1) for 1 h at 37° C. Proteolytic digestion was terminated by the addition of PMSF to a final concentration of 2 mM. 40 μg of the digested cell lysate was mixed with an equal volume of 2× non-reducing SDS sample buffer, boiled for 5 minutes, cleared by centrifugation and resolved by SDS/PAGE (14%). Samples were electro-blotted onto PVDF membranes and blocked with 5% (w/v) nonfat milk protein in calcium and magnesium-free PBS. PrP was detected using 0.5 μg/ml D18 antibody crosslinked to pre-activated amine reactive horseradish peroxidase (Pierce). Blots were developed with enhanced chemiluminescence (ECL) reagent (Amersham) for 1 min, and exposed to ECL Hypermax film (Amersham). Denistometric scanning of PrP^{Sc} bands was performed with Chemi Imager 4000 Low Light Imaging System using AlphaEase software version 3.3e (Alpha Innotech, Calif.). Apparent amounts of PrP^{Sc} (denstometric units) were plotted as a percentage of PrP^{Sc} found in equivalent untreated ScN2a cell cultures on the day of harvest.

Bioassays. Antibody-treated (60 days) and untreated ScN2a cells were resuspended in 1 ml of PBS. A 30 μl volume of the cell resuspension was inoculated intracerebrally into groups of 10 CD-1 Swiss mice. Mice were scored daily for early and late onset of clinical signs of scrapie. Infectious titers of mouse prions in CD-1 Swiss mice were derived by use of the equation Log T=1.52 +[Log D] +(185−Y)/12.66; where T is ID₅₀ units/ml; D is the dilution, defined as the fractional dilution of the diluted sample; and Y is the mean interval from inoculation to onset of terminal illness in days (Butler, D. A. et al. Scrapie-infected murine neuroblastoma cells produce protease-resistant prion proteins. J. Virol. 62, 1558-1564 (1988)).

Flow cytometry. ScN2a cells were resuspended with cell dissociation buffer (GIBCO BRL) and washed twice with FACS buffer (MEM, 5% (v/v) cell dissociation buffer and 2% (v/v) FBS). Increasing concentrations of PrP-specific Fabs were added to aliquots of 10⁶ cells. After 15 min incubation at room temperature cells were washed 4 times with FACS buffer and stained for 15 min at room temperature with a 1:200 dilution in FACS buffer of fluorescein isothiocyanate (FITC)-conjugated goat anti-human IgG Fab (Jackson Immunologicals). Cells were then washed three times in FACS buffer, fixed with freshly prepared 0.4% (v/v) paraformaldehyde and analyzed using the Becton Dickenson FACscan instrument.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method of preventing infection with PrP^{Sc} protein, comprising the steps of:
contacting cells with a formulation of molecules which bind to a plurality of epitopes on PrP^{C} protein; and
allowing the molecules to remain in contact with the cells for a period of time and under conditions such that binding occurs between the molecules and a plurality of epitopes on PrP^{C} whereby the molecules hinder a change in conformation from PrP^{C} protein to PrP^{Sc} protein.

2. The method of claim 1, wherein the molecules are antibodies which bind to PrP^{C}.

3. The method of claim 1, wherein the molecules comprise Fab fragments selected from the group consisting of D13, D18, R1 and R2.

4. A method of clearing a disease conformation of a protein from a cell, comprising the steps of:
contacting a cell with a formulation of molecules, wherein the cell is infected with a protein which assumes a first non-disease conformation and wherein both conformations are present in the cell,
allowing the molecules to remain in contact with the cell for a period of time and under conditions such that binding occurs between the molecules and a plurality of epitopes on the first conformation of the protein whereby conversion to the second conformation of the protein is prevented for a period of time sufficiently long to allow the cell to clear protein in the second conformation from the cell.

5. The method of claim 4, wherein the protein is a PrP protein, the first conformation is PrP^{C} and the second conformation is PrP^{Sc}.

6. The method of claim 5, wherein the molecules are antibodies which bind PrP^{C}.

7. The method of claim 5, wherein the molecules comprise Fab fragments selected from the group consisting of D13, D18, R1 and R2.

8. A method of assaying for molecules which hinder binding to PrP^{C}, comprising the steps of:
providing PrP^{C} molecules;
providing a test compound;
providing an antibody which binds PrP^{C};
allowing the test compound to interact with the PrP^{C} molecules and antibody for a period of time and under conditions such that binding of the antibody to PrP^{C} molecules would be expected;
determining a level of binding of the PrP^{C} to the antibody; and
calculating the effect of the test compound to effect binding between the antibody and PrP^{C} molecules.

9. The method of claim 8, wherein the antibody is selected from the group consisting of D18 and D13.

10. The method of claim 8, wherein the antibody is bound to a support surface and the PrP^{C} and test compound are provided in a solution or a suspension.

11. The method of claim 8, wherein the PrP^{C} is bound to a support and the antibody and the test compound are provided in a solution or a suspension.

12. A method of treatment, comprising:
administering to a subject a therapeutically effective amount of an antibody which binds to PrP^{C} wherein the antibody is present in a pharmaceutically acceptable carrier.

13. The method of claim 12, wherein the antibody is selected from the group consisting of D13 and D18.

14. A method of treatment, comprising:
administering to a subject an antigen which results in the generation of antibodies by the subject wherein the antibodies bind PrP^{C}.

15. A composition for preventing infection with PrP^{Sc} protein, comprising:
a formulation comprising a pharmaceutically acceptable carrier and molecules which bind to a plurality of epitopes on PrP^{C} protein which molecules remain bound to the epitopes for a period of time and under conditions such that binding occurs between the molecules and a plurality of epitopes on PrP^{C} whereby the molecules hinder a change in conformation from PrP^{C} protein to PrP^{Sc} protein.

16. The composition of claim 15, wherein the molecules are antibodies which bind epitopes on PrP^{C}.

17. The composition of claim 15, wherein the molecules comprise Fab fragments selected from the group consisting of D13, D18, R1 and R2.

18. A composition for clearing a disease conformation of a protein from a cell, comprising:
a formulation comprising a pharmaceutically acceptable carrier and molecules which bind a plurality of epitopes on a first conformation of a protein whereby conversion to a second conformation of the protein is prevented for a period of time sufficiently long to allow a cell to clear protein in the second conformation from the cell.

19. The composition of claim 18, wherein the protein is a PrP protein, the first conformation is PrP^{C} and the second conformation is PrP^{Sc}.

20. The composition of claim 19, wherein the molecules are antibodies which bind PrP^{C}.

21. The composition of claim 19, wherein the molecules comprise Fab fragments selected from the group consisting of D13, D18, R1 and R2.


SEQUENCE LISTING (4 sequences)

SEQ ID NO: 1 - protein, 254 aa, mus musculus
MANLGYWLLALFVTMWTDVGLCKKRPKPGGWNTGGSRYPGQGSPGGNRYPPQGGTWGQPHGGGWGQPHGGSWGQPHGGSWGQPHGGGWGQGGGTHNQWNKPSKPKTNLKHVAGAAAAGAVVGGLGGYMLGSAMSRPMIHFGNDWEDRYYRENMYRYPNQVYYRPVDQYSNQNNFVHDCVNITIKQHTVTTTTKGENFTETDVKMMERVVEQMCVTQYQKESQAYYDGRRSSSTVLFSSPPVILLISFLIFLIVG

SEQ ID NO: 2 - protein, 253 aa, homo sapiens
MANLGCWMLVLFVATWSDLGLCKKRPKPGGWNTGGSRYPGQGSPGGNRYPPQGGGGWGQPHGGGWGQPHGGGWGQPHGGGWGQPHGGGWGQGGGTHSQWNKPSKPKTNMKHMAGAAAAGAVVGGLGGYMLGSAMSRPIIHFGSDYEDRYYRENMHRYPNQVYYRPMDEYSNQNNFVHDCVNITIKQHTVTTTTKGENFTETDVKMMERVVEQMCITQYERESQAYYQRGSSMVLFSSPPVILLISFLIFLIVG

SEQ ID NO: 3 - protein, 263 aa, bovine
MVKSHIGSWILVLFVAMWSDVGLCKKRPKPGGWNTGGSRYPGQGSPGGNRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDQYSNQNNFVHDCVNITVKEHTVTTTTKGENFTETDIKMMERVVEQMCVTQYQKESQAYYDQGASVILFSSPPVILLISFLIFLIVG

SEQ ID NO: 4 - protein, 255 aa, sheep
MVKSHIGSWILVLFVAMWSDVGLCKKRPKPGGWNTGGSRYPGQGSPGGNRYPPQGGGGWGQPHGGGWGQPHGGGWGQPHGGSWGQPHGGGGWGQGGSHSQWNKPSKPKTNMKHVAGAAAAGAVVGGLGGYMLGSAMSRPLIHFGNDYEDRYYRENMYRYPNQVYYRPVDQYSNQNNFVHDCVNITVKQHTVTTTTKGENFTETDIKIMERVVEQMCITQYQRESQAYYQRGASVILFSSPPVILLISFLIFLIVG